(19) United States
(12) Patent Application Publication
(10) Pub. No.: US 2021/0330205 A1
(43) Pub. Date: Oct. 28, 2021

(54) APPARATUS, COMPUTER-ACCESSIBLE MEDIUM, SYSTEM AND METHOD FOR DETECTION, ANALYSIS AND USE OF FETAL HEART RATE AND MOVEMENT

(71) Applicants: New York University, New York, NY (US); Stevens Institute of Technology, Hoboken, NJ (US)

(72) Inventors: NEGAR TAVASSOLIAN, Hoboken, NJ (US); CHENXI YANG, Jersey City, NJ (US); BRUCE YOUNG, New York, NY (US)

(73) Assignees: New York University; Stevens Institute of Technology

(21) Appl. No.: 17/237,403

(22) Filed: Apr. 22, 2021

Related U.S. Application Data
(60) Provisional application No. 63/013,692, filed on Apr. 22, 2020.

(51) Int. Cl. A61B 5/024; A61B 5/344; A61B 5/11

(52) U.S. Cl. CPC A61B 5/02411; A61B 5/344; A61B 2562/0204; A61B 2562/0219; A61B 5/11

(57) ABSTRACT

Exemplary embodiments of the present invention provide for an apparatus, computer-accessible medium, system and method for detection, analysis and use of fetal heart rate and movement. In accordance with certain exemplary embodiments of the present disclosure, an exemplary system can include: at least one electrocardiogram sensor providing first signals or information regarding the at least one subject; a plurality of inertial measurement units providing second signals or information regarding the at least one subject; a plurality of acoustic sensors providing third signals or information regarding the at least one subject; and a processor, wherein the processor is configured to determine data regarding the fetal heart rate based on the first, second and third signals or information.

### CROSS REFERENCE TO RELATED APPLICATION(S)

This application relates to and claims priority from U.S. Provisional Patent Application Ser. No. 63/013,692, filed Apr. 22, 2020, the disclosure of which is incorporated herein by reference in their entirety.

### FIELD OF THE DISCLOSURE

The present disclosure relates generally to the detection of fetal heart rate and fetal movement, and more specifically, to exemplary embodiments of exemplary apparatus, computer-accessible medium, system and method for detecting, analysis and use of fetal heart rate and movement.

### BACKGROUND INFORMATION

Recent reports indicate that objective and continuous monitoring of fetal heart rate (FHR) and fetal movement (FMV) could identify fetal compromise with high reliabilities and decrease stillbirth through time-sensitive management. In the United States alone, an average of 26,000 cases of perinatal mortality occur per year. In the United Kingdom, 29% of stillbirths occur in the absence of complicating factors. This can result in an urgent desire for a continuous fetal monitoring (CFM) system that can keep track of both the FHR and FMV. FHR and FMV are the only viable biologic signs that can be continuously monitored and assessed. The fetal heart rate variation (HRV) decreases dramatically days before stillbirth, until a loss of HR and HRV occurs several hours before the actual stillbirth. Baseline and acceleration abnormalities have also been reported in the cases of fetal cardiovascular and neural system diseases.

Fetal fardiotocography (fCTG) and fetal electrocardiography (fECG) are wearable technologies which are widely employed in current clinical settings for monitoring FHR and FMV. The current fCTG technology uses external Doppler ultrasound sensors to monitor the FHR and the activity of the uterine muscle. Despite their extensive use, current ultrasound sensors have drawbacks. For example, they may be harmful to the fetus if used over extended periods of time due to teratogenic or fetotoxic effects from ultrasonic heating of fetal tissues. This drawback limits their potential to be used as wearable and continuous home-based monitors. Further, the FDA has recommended that current commercial Doppler ultrasound fetal monitors be avoided outside the clinic due to low accuracies as well as unpredictable risks due to unguided usage.

Current fECG technology uses multiple multi-lead electrodes to detect FHR and monitors the rotation of the fetus to estimate FMV. In particular, the current technology records multi-lead abdominal ECG signals. Then, by wide-spreading electrodes along the abdomen, the fECG can be extracted by fusing multiple ECG recordings. As such, the size of the device incorporating the multi-lead electrodes is relatively large, usually in the form factor of a wearable belt or brace around the abdomen. Further, the signal quality of abdominal fECG highly depends on the position of the electrodes with respect to the fetus, and therefore the accuracy is highly variable.

Further, other technologies can also be used to detect FHR in a wearable setup. For example, fetal photoplethysmography (fPPG) and fetal phonocardiography (fPCG) sense FHR by detecting optical and acoustic signals, respectively. However, these two technologies can detect FHR with lower accuracies and reliabilities compared to fECG. They also lack the capability of monitoring FMV. For example, current fPCG monitors are sensitive to the location of the heart, which can change when the fetus rotates inside the womb. As such, the current fPCG monitors need to be actively relocated anytime a new measurement is taken so that the sensor is as close to the fetal heart as possible.

Further, although current multi-modality system can fuse ECG and acoustic information to extract FHR, they lack the capability of detecting FMV.

Thus, it may be beneficial to provide exemplary apparatus, computer-accessible medium, system and method for detection, analysis and use of fetal heart rate and movement thereof which can overcome at least some of the deficiencies described herein above.

### SUMMARY OF EXEMPLARY EMBODIMENTS

Exemplary embodiments of the present invention provide for a system consisting of a wearable device and software for recording fetal heart rate (FHR), fetal movement (FMV), and monitoring fetal health status. The exemplary wearable systems can use an array of inertial measurement units (IMUs), an array of beamforming acoustic sensors, and a single-lead electrocardiogram (ECG) sensor. These sensors can record vibrations, PCG signals, and ECG signals from the surface of the abdomen of the pregnant subject, respectively. The exemplary systems can fuse information from all sensors and extract the FHR and FMV. In this exemplary way, the exemplary system can be used for the early detection of fetal abnormalities, such as, e.g., stillbirth in a ubiquitous, noninvasive, home-based healthcare setup for those with high-risk pregnancies, as well as those with non-high-risk pregnancies as a general self-healthcare product.

An exemplary apparatus, computer-accessible medium, system and method for detection, analysis and use of FHR and FMV can include: at least one ECG sensor providing first signals or information regarding the at least one subject; a plurality of IMUs providing second signals or information regarding the at least one subject; a plurality of acoustic sensors providing third signals or information regarding the at least one subject; and a processor, wherein the processor is configured to determine data regarding the FHR based on the first, second and third signals or information.

In some exemplary embodiments of the present disclosure, the at least one ECG sensor can be a single-lead ECG sensor. Further, the plurality of acoustic sensors can be configured to provide beam-formed signals to the at least one subject.

According to further exemplary embodiments of the present disclosure, method, system and non-transitory computer-accessible can be provided for forwarding an excited fluorescence radiation, which can, e.g., using at least one electrocardiogram (ECG) sensor, obtain first signals or information regarding the subject (s); using a plurality of inertial measurement units (IMUs), obtain second signals or information regarding the subject(s); with a plurality of acoustic sensors, obtain third signals or information regarding the subject(s); and using one or more computer processors, determine data regarding the FHR based on the first, second and third signals or information.

Various exemplary embodiments of the present disclosure can provide the following advantages: (i) instead of multi-lead electrodes, single-lead ECG electrodes are used, resulting in a smaller form factor, (ii) unlike current fPCG sensors, the exemplary system uses beamforming technology with a sensor array to focus on the acoustic signal coming from the direction of the fetal heart, resulting in a higher flexibility in attaching the sensor to the surface of the abdomen without the need of finding the optimal abdominal location, (iii) unlike current multi-modality systems, the exemplary system can monitor both FHR and FMV by fusing ECG, acoustic, and inertial sensors, (iv) and the FHR and FMV can be monitored with passive sensor systems, thereby avoiding the side effects associated with an active sensor systems such as ultrasound.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying

Figures showing illustrative embodiments of the present disclosure, in which:

FIG. 1 is an exemplary diagram of a wearable hardware platform in accordance with certain exemplary embodiments of the present disclosure;

FIG. 2 is a flow diagram of a signal processing method in accordance with certain exemplary embodiments of the present disclosure;

FIG. 3A is an exemplary image of experimental setup and environment in accordance with certain exemplary embodiments of the present disclosure;

FIG. 3B is an exemplary sensor setup for the experiment illustrated in FIG. 3A in accordance with certain exemplary embodiments of the present disclosure;

FIG. 4 is a graph of a representative filtered SCG signal in accordance with certain exemplary embodiments of the present disclosure;

FIG. 5A is a set of exemplary continuous wavelet transform plots for a first sensor illustrated in FIG. 3B in accordance with certain exemplary embodiments of the present disclosure;

FIG. 5B is a set of exemplary continuous wavelet transform plots for a second sensor illustrated in FIG. 3B in accordance with certain exemplary embodiments of the present disclosure;

FIG. 5C is a set of exemplary continuous wavelet transform plots for a third sensor illustrated in FIG. 3B in accordance with certain exemplary embodiments of the present disclosure;

FIG. 5D is a set of exemplary fused continuous wavelet transform plots for the sensors illustrated in FIG. 3B in accordance with certain exemplary embodiments of the present disclosure;

FIG. 6 is a graph of an exemplary cepstrum of seismo-cardiogram recordings in accordance with certain exemplary embodiments of the present disclosure; and

FIG. 7 is an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

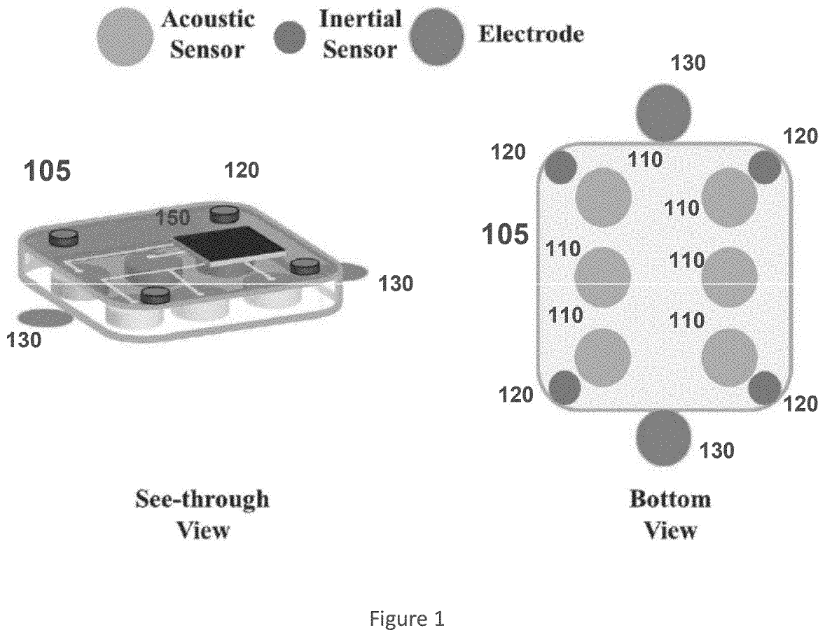

According to an exemplary embodiment of the present disclosure, an exemplary system can be provided which can have a few parts/portions thereof. The first part of the exemplary system can be or include a wearable wireless hardware platform consisting of sensor arrays that collect recordings with precise timing for synchronization. The wearable device can be placed on the surface of a subject's abdomen. An illustration of an exemplary system which shows an exemplary layout of the sensors is provided in FIG. 1. As depicted in FIG. 1, the exemplary system 105 can include an array of acoustic sensors (orange circles) 110, an array of inertial sensors (gray circles) 120, e.g., IMUs, and a set of single-lead dry electrodes (blue circles) 130. According to an exemplary embodiment of the present disclosure, the IMUs 120 can include accelerometers and gyroscopes. Further, the acoustic sensors 110 can include beamforming technology, thereby facilitating the acoustic sensors 110 to focus on the acoustic signal coming from the direction of the fetal heart. According to an exemplary embodiment of the present disclosure, the beamforming acoustic sensors can be located at a set distance away from each other. Further, IMUs can also be located at a set distance away from each other.

The exemplary system 105 can also contain integrated circuit chips (ICs), such as, e.g., a microcontroller unit (MCU) and/or low-power field programmable gate arrays (FPGA), wireless communication chips, as well as other active and passive peripheral components such as resistors, connectors, capacitors, power ICs and printed circuit boards (PCB), represented by the black and the light-color squares in FIG. 1 (on the left side thereof) 150. Further, a rechargeable battery, which is not shown in the figure, can also be included. According to an exemplary embodiment of the present disclosure, the rechargeable battery(not shown) can be connected to an energy harvester. For example, the rechargeable battery can be charged based on one of (i) the motion of the wearable device, (ii) the heat generated from a body wearing the device, (iii) radio frequency signals from the air.

According to an exemplary embodiment of the present disclosure, the IMUs can pick up/detect vibrations that are induced by fetal heartbeat and movements from the abdominal wall. In particular, the accelerometers can measure a seismo-cardiogram (SCG) signal, while the gyroscopes can measure the gyro-cardiogram (GCG) signal. The SCG signal can be defined as the heartbeat-induced micro-vibrations of the chest wall. The GCG signal corresponds to the rotational components of heart-induced chest vibrations. The acoustic sensors can collect the acoustic information caused by fetal heartbeat, which is also known as fetal phonocardiogram. The electrodes 130 can record the abdominal electrocardiogram (aECG) signal, which can be the electric potential generated by maternal and fetal cardiac activities on the abdominal wall. Based on this gathered data, the FHR and FMV can be derived. According to an exemplary embodiment, the FHR can be presented in the unit of beats per minute (BPM). Further, the FHR can be derived continuously by fusing the raw signals from the IMU sensors, acoustic sensors, and aECG electrodes. Further, the maternal ECG components of the aECG can be removed during the fusion so that the fetal ECG can be extracted. Additional information can be inferred from FHR such as baseline FHR, FHR variability, FHR acceleration, and FHR deceleration. These exemplary parameters can used to determine the cardiac wellness of the fetus. Further, the

FMV can be detected via the IMUs. The strength, duration, and repetition frequency of the movements can be recorded by the system and analyzed to determine the activity level of the fetus. The data can then be transmitted wirelessly to a personal computer or smartphone application, which can then evaluate the wellbeing of the fetus and detect potential fetal abnormalities.

According to an exemplary embodiment of the present disclosure, another part/portion of the exemplary system can be a signal processing software which can include the embedded firmware in the wearable device as well as the software on the smartphone or personal computer (PC) application. The embedded software can manage the sensors and conducts signal processing and noise reduction. Useful pieces of data (for example, peak timing and amplitude information) can then be compressed and wirelessly transferred to the smartphone or PC application in real time. In an exemplary embodiment of the present disclosure, the application can store, log, store and analyze the data and provide feedback to the user and/or proper notice to the physician via internet connections.

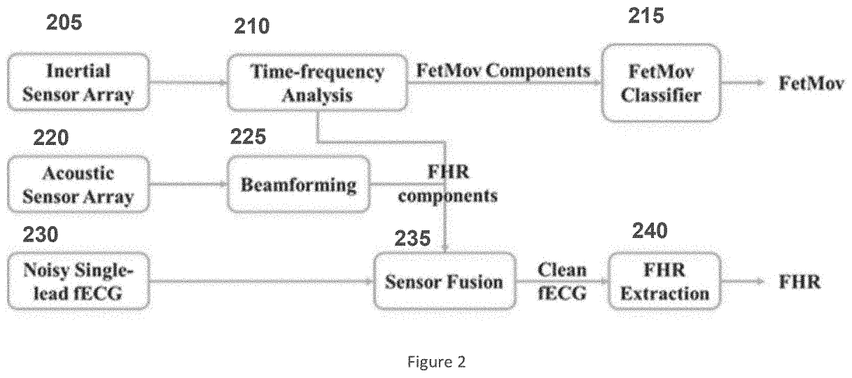

FIG. 2 illustrates a flow diagram of a signal processing method according to an exemplary embodiment of the present disclosure. In particular, FIG. 2 illustrates exemplary flow of FHR and FMV extraction from, e.g., the three modalities. First, IMU inertial sensor array cam be provided in in 205. Then, the information from the IMU sensor array can be processed by a time-frequency analysis procedure in 210. The exemplary time-frequency analysis procedure can separate the components that are related to FMV from the components related to FHR. The FMV components can be transmitted to a classifier that detects fetal movements in 215.

The FHR components from the IMU sensors can be fed into an exemplary sensor fusion procedure in 235, together with the FHR components from the acoustic beamforming output in 225, which are provided from acoustic sensor array(s) provided in 220. The exemplary sensor fusion procedure can clean the noisy single-lead fECG using these FHR components, which can generates clean fECG recordings. The FHR metrics can then be calculated or otherwise determined from the fECG recordings in 240.

Exemplary Procedure for Extracting FHR Components from the IMUs

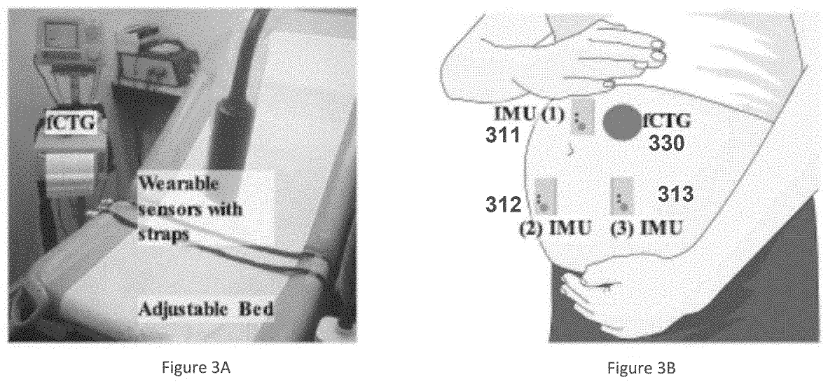

Exemplary Experimental Setup—FIGS. 3A and 3B illustrate an exemplary experimental setup. In particular, FIG. 3A shows an image of the experimental setup and environment, and FIG. 3B illustrates an exemplary sensor setup including 3 IMU sensor nodes 311-313 and a reference fCTG ultrasound sensor probe 330, which can be recorded, e.g., by a FETALGARD Lite system (Version 1.02, using US1 channel). For example, as depicted in FIG. 3B, three (and which can be any number of two or more) wearable IMU sensor nodes 310 can be attached to an abdominal wall with elastic straps. One sensor node 311 can be placed at the center of the upper abdominal wall (e.g., sensor (1) 311 shown in FIG. 3B). This sensor 311 can be close to the reference fCTG ultrasound probe. The remaining two sensors (e.g., sensors (2) and (3) 312, 313 shown in FIG. 3B) can be attached on the lower part of the abdominal wall at symmetric locations. According to an exemplary embodiment of the present disclosure, the accelerometer and gyroscope in the IMUS can have ranges of ±2 g and ±250 degrees per second (DPS), respectively, and all the sensor recordings can be sampled at a sampling rate of 512 Hz. Data can be stored in a memory card on the IMU sensor node and then transmitted to a computer for digital signal processing. Further, the reference CTG and the sensor recordings can be synchronized by cross-checking their timestamps. All the data are then imported into computer software, e.g., MATLAB® (R2018), for further processing.

Experimental Protocol—The experiment can be conducted in an fCTG examination room with an adjustable bed as shown in FIG. 3A. The experiment can include, e.g., three steps. During the first step, the subjects can be placed in a supine position for five minutes. The subjects can then change to a seated position, and the monitoring continues for another five minutes. Afterwards, the subjects can stand up and are monitored for an additional five minutes.

Exemplary Signal Processing Method—Pre-Filtering—In the exemplary axis system of the IMU sensors, the z-axis refers to the dorso-ventral direction of the body. The z-axis of the SCG signal can be evaluated first before fusing the information from multiple axes. For the GCG modality, the y-axis rotation signal can be selected due to the higher quality for this axis.

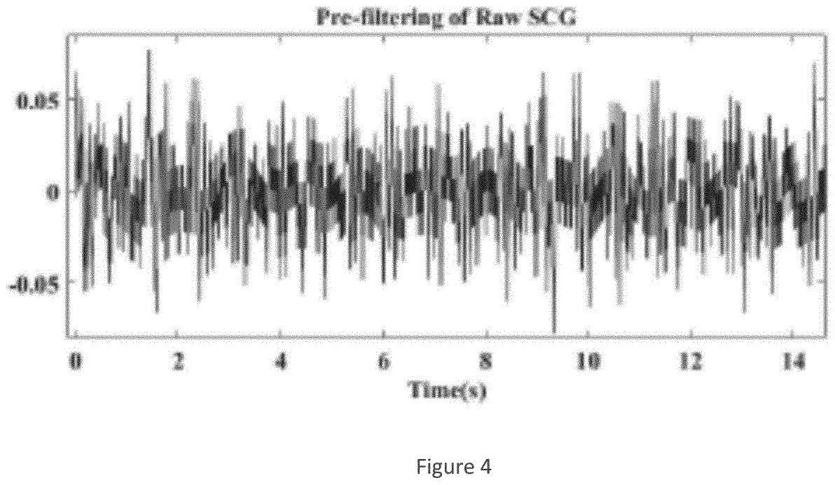

Exemplary SCG and GCG recordings from the corresponding axes can be band-pass filtered to focus on the desired frequency components. A zero-phase infinite impulse response (IIR) filter that passes from 0.8 Hz to 50 Hz can be used to pre-filter the SCG waveforms. FIG. 4 shows a representative filtered SCG signal. The amplitude of the signal can be quite small, suggesting a weak vibration from the abdominal surface. The observation from GCG is similar to that from SCG. Therefore, the information from all three sensors can be fused to enhance the signal quality of SCG and GCG separately. The z-axis SCG from three sensors can be fused, and the y-axis GCG from the three sensors can be fused, as described in the following section.

Exemplary Signal Processing Method—Signal Fusion of Multiple Sensors—The ensemble of the recordings in time domain is not suitable for analysis since the axes of the signals from different sensors are misaligned due to the abdominal wall being a curved surface.

Therefore, the vibration components from different sensors do not align in the same direction and hence the direct summation of the amplitudes would be misleading. In this regard, the signals can be processed using time-frequency analysis based on continuous wavelet transform (CWT). CWT converts the signal into the time-frequency domain, so that the desired frequency components can be fused without losing the time-domain variations. The pre-processed SCG and GCG signals can be converted by CWT with a Morse wavelet, as provided below:

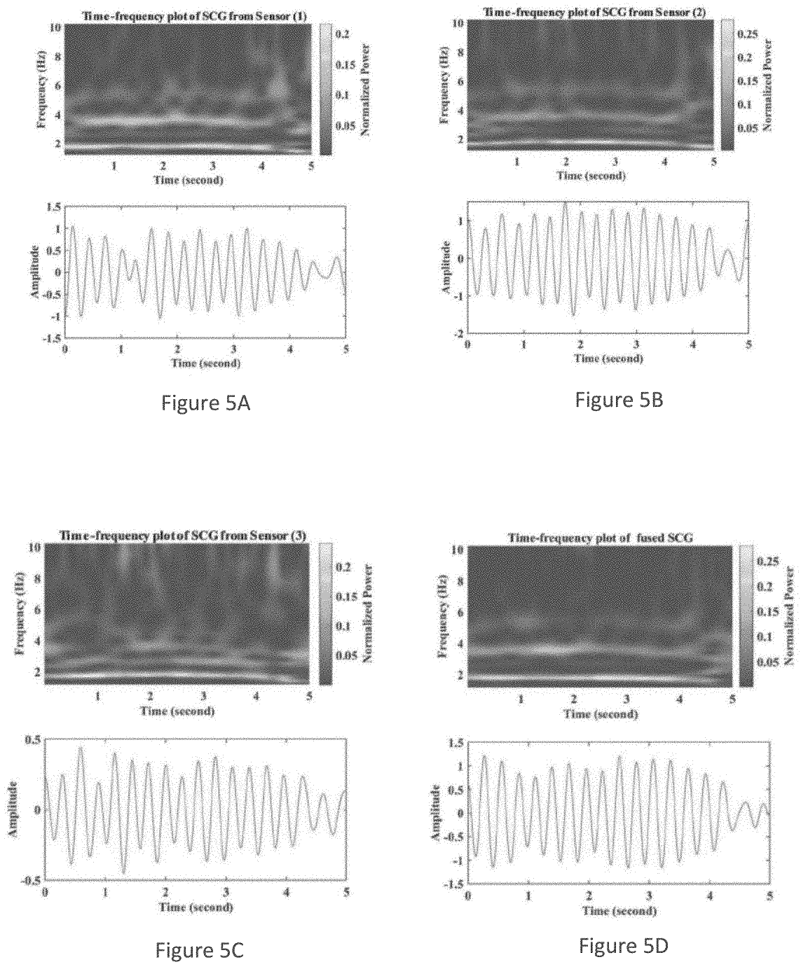

Ψ_{P,γ}(ω)=U (ω) α_{p,65} ω^{(P}^{_{2/γ)}}e^{-ω}^{_{γ}},

where P is the time-bandwidth product and γ is the symmetry parameter. In this regard, γ can be set to 3 and P can be set 120. The dominant frequency band of the FHR signals is located based on the power distribution of the CWT coefficients. An averaging function can then fuse the CWT coefficients from the corresponding frequency band of the three sensors. Then, a frequency-selective inverse CWT can be conducted to reconstruct a signal that represents FHR. The exemplary results from a representative SCG segment are shown in FIGS. 5A to 5D. The top plot in each figure shows the heat map of the CWT, while the bottom plot illustrates the frequency-selective inverse CWT. The exemplary results from each sensor are illustrated in FIG. 5A to 5C, followed by the results from the fused CWT in FIG. 5D. For example, exemplary plots of FIG. 5A corresponds to sensor (1) 311 shown in FIG. 3B, exemplary plots of FIG. 5B corresponds to sensor (2) 312 shown in FIG. 3B, and exemplary plots of FIG. 5C corresponds to sensor (3) 313 shown in FIG. 3B. It can be seen that there are differences among the heat maps of FIGS. 5A-5C, especially in the dominant band of the vibration signal (1-5 Hz). For instance, the frequency-selective inverse CWT from sensor (1) 311 shows several attenuated peaks compared to the results from the fused CWT in FIG. 5D. A similar observation can also be found from the inverse CWT results of sensor (3) 313 (i.e., FIG. 5C). The exemplary results from sensor (2) 312 shown in FIG. 5B are comparable to the fused results. In summary, signal stability can be improved after fusion.

Signal Processing Method—FHR Extraction—The spectrums of the fused waveforms can be analyzed by the cepstrum method. The cepstrum is defined as the inverse Fourier transform of the real logarithm of the magnitude of the Fourier transform of a time-domain sequence. The exemplary method can be presented in the equation below:

Csig=real(F⁻¹ {log(F|(x)|)}.

In the above equation, x represents the fused waveform from CWT shown in the exemplary plots of FIG. 5D. The cepstrum is close in definition with the autocorrelation function, which is indexed also by lag time, with the difference that the inverse Fourier transform is taken from the squared spectrum (i.e., power spectral density) instead of the logarithm of the spectrum. The FHR can then be presented as the periodicity in the spectrum, shown as a peak value in the cepstrum located at the corresponding lag time value.

Based on the sensor fusion framework described above, the FHR can then be extracted from the recordings. The sliding window for CWT can be set to 5 seconds to approximate the averaging process. The FHR recordings from the reference fCTG can range between 120 and 180 BPM. Therefore, the FHR can be targeted within this range. The highest peak that locates in the range from 0.33 to 0.5 seconds (2 Hz to 3 Hz in repeating frequency) can be identified as the FHR period.

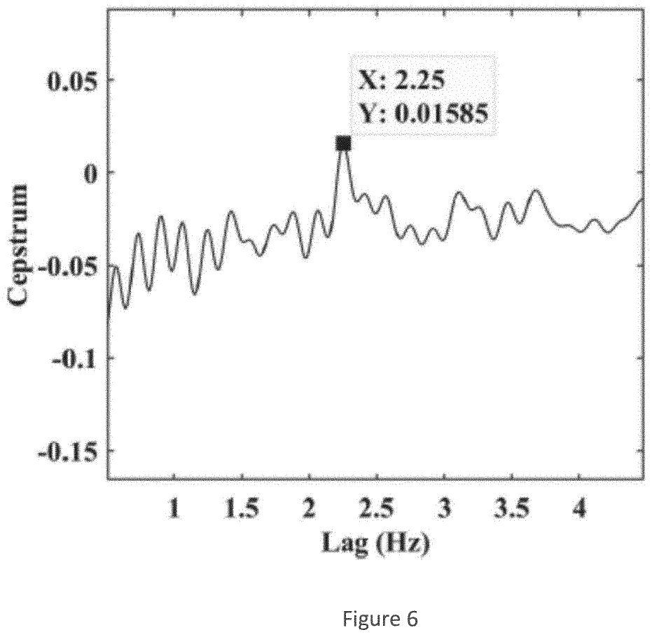

FIG. 6 shows an exemplary graph of the cepstrum of a representative section from the seismo-cardiogram (SCG) recordings, according to an exemplary embodiment of the present disclosure. It can be seen from FIG. 6 that there is a detected peak with a lag at 2.25 Hz, highlighted with a black square. As a result, the FHR of this interval is 2.25×60=135 BPM.

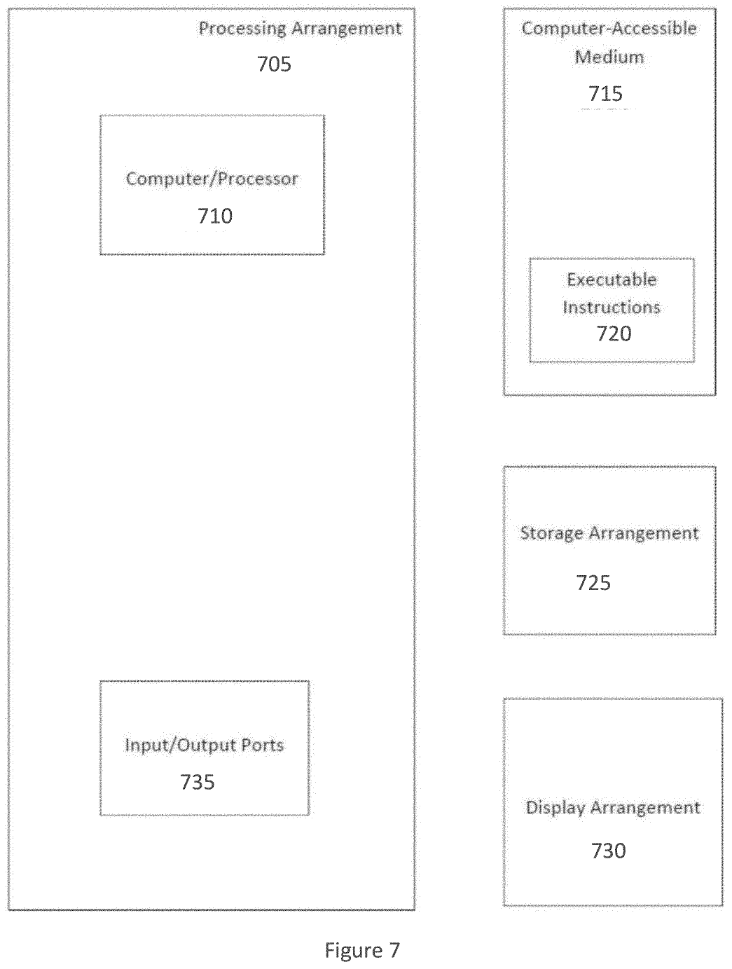

FIG. 7 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 705. Such processing/computing arrangement 705 can be, for example entirely or a part of, or include, but not limited to, a computer/processor 710 that can include, for example one or more microprocessors, and use instructions stored on a computer-accessible medium (e.g., RAM, ROM, hard drive, or other storage device).

As shown in FIG. 7, for example a computer-accessible medium 715 (e.g., as described herein above, a storage device such as a hard disk, floppy disk, memory stick, CD-ROM, RAM, ROM, etc., or a collection thereof) can be provided (e.g., in communication with the processing arrangement 705). The computer-accessible medium 715 can contain executable instructions 720 thereon. In addition or alternatively, a storage arrangement 725 can be provided separately from the computer-accessible medium 715, which can provide the instructions to the processing arrangement 705 so as to configure the processing arrangement to execute certain exemplary procedures, processes, and methods, as described herein above, for example.

Further, the exemplary processing arrangement 705 can be provided with or include an input/output ports 735, which can include, for example a wired network, a wireless network, the internet, an intranet, a data collection probe, a sensor, etc. As shown in FIG. 7, the exemplary processing arrangement 705 can be in communication with an exemplary display arrangement 730, which, according to certain exemplary embodiments of the present disclosure, can be a touch-screen configured for inputting information to the processing arrangement in addition to outputting information from the processing arrangement, for example. Further, the exemplary display arrangement 730 and/or a storage arrangement 725 can be used to display and/or store data in a user-accessible format and/or user-readable format.

According to an exemplary embodiment of the present disclosure, the exemplary system can be used to detect FHR and/or FMV at 28 weeks of the gestational life of a fetus.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference, in their entireties:

1. M. F. MacDorman, S. E. Kirmeyer, and E. C. Wilson, “Fetal and perinatal mortality, United States, 2006,” Nat. Vital Statist. Rep., vol. 60, no. 8, pp. 1-22, 2012.

2. F. J. Korteweg et al., “A placental cause of intra-uterine fetal death depends on the perinatal mortality classification system used,” Placenta, vol. 29, no. 1, pp. 71-80, 2008.

3. V. Flenady et al., “An evaluation of classification systems for stillbirth,” BMC Pregnancy Childbirth, vol. 9, p. 24, Jun. 2009.

4. Perinatal Mortality 2009, CMACE, London, U.K., 2011.

5. R. L. Goldenberg et al., “Stillbirths: The vision for 2020,” Lancet, vol. 377, pp. 1798-1805, May 2011.

6. R. Brown, J. H. B. Wijekoon, A. Fernando, E. D. Johnstone, and A. E. P. Heazell, “Continuous objective recording of fetal heart rate and fetal movements could reliably identify fetal compromise, which could reduce stillbirth rates by facilitating timely management,” Med. Hypotheses, vol. 83, no. 3, pp. 410-417, 2014.

7. R. Sameni and G. D. Clifford, “A review of fetal ECG signal processing; Issues and promising directions,” Open Pacing, Electrophysiol. Therapy vol. 3, p. 4, Jan. 2010.

8. S. P. von Steinburg et al., “What is the ‘normal’ fetal heart rate?” Pier J., vol. 1, Jun. 2013. [Online]. Available: https://peerj.com/articles/82/.

9. J. F. Frøen et al., “Restricted fetal growth in sudden intrauterine unexplained death,” Acta Obstetricia Gynecologica Scandinavica, vol. 83, no. 9, pp. 801-807, 2004.

10. R. K. Freeman, Fetal Heart Rate Monitoring. Philadelphia, Pa., USA: Lippincott Williams & Wilkins, 2012.

11. E. W. Abdulhay, R. J. Oweis, A. M. Alhaddad, F. N. Sublaban, M. A. Radwan, and H. M. Almasaeed, “Review article: Non-invasive fetal heart rate monitoring techniques,” Biomed. Sci. Eng., vol. 2, no. 3, pp. 53-67, 2014.

12. B. K. Young, H. N. Weinstein, H. M. Hochberg, and M. E. George, “Observations in perinatal heart rate monitoring. I. A quantitative method of describing baseline variability of the fetal heart rate,” J Reproductive Med., vol. 20, no. 4, pp. 205-212, 1978.

13. K. Nicolaides et al. (2002). Doppler in Obstetrics. [Online]. Available: http://www/fetalmedicine/com/fmf/Doppler%20in%20Obstetrics.pdf

14. Avoid Fetal ‘Keepsake ’ Images, Heartbeat Monitors. Accessed: Jul. 30, 2019. [Online]. Available: https://www.fda.gov/ForConsumers/ConsumerUpdates/ucm095508.htm

15. R. Vullings, C. H. L. Peters, M. Mischi, S. G. Oei, and J. W. M. Bergmans, “Fetal movement quantification by fetal vectorcardiography: A preliminary study,” in Proc. 30th Annu. Int. Conf. IEEE Eng. Med. Biol. Soc., Aug. 2008, pp. 1056-1059.

16. E. M. Graatsma, “Monitoring of fetal heart rate and uterine activity,” Ph.D. dissertation, Utrecht Univ., Utrecht, The Netherlands, 2010.

17. E. M. Graatsma, B. C. Jacod, L. A. J. van Egmond, E. J. H. Mulder, and G. H. A. Visser, “Fetal electrocardiography: Feasibility of long-term fetal heart rate recordings,” BJOG, Int. J. Obstetrics Gynaecol., vol. 116, no. 2, pp. 334-338, 2009.

18. Monica. Introducing the Monica AN24. Accessed: Jul. 30, 2019. [Online]. Available: http://www.monicahealthcare.com/products/

19. Avalon Fetal Monitor. [Online]. Accessed: Jul. 30, 2019. Available: https://www.usa.philips.com/healthcare/resources/landing/avalon

20. A. Fanelli et al., “Prototype of a wearable system for remote fetal monitoring during pregnancy,” in Proc. Annu. Int. Conf. IEEE Eng. Med. Biol., Sep. 2010, pp. 5815-5818.

21. T. H. Sree, M. Garimella, A. Bandari, and I. Patel, “Microcontroller based fetal heart rate monitoring using intelligent biosystem,” in Proc. 3rd Int. Conf. Electron., Biomed. Eng. Appl. (ICEBEA), Singapore, Apr. 2013, pp. 152-156.

22. K. B. Gan, E. Zahedi, and M. A. M. Ali, “Investigation of optical detection strategies for transabdominal fetal heart rate detection using three-layered tissue model and Monte Carlo simulation,” Optica Appl., vol. 41, no. 4, pp. 885-896, 2011.

23. R. Martinek, “A phonocardiographic-based fiber-optic sensor and adaptive filtering system for noninvasive continuous fetal heart rate monitoring,” Sensors, vol. 17, no. 4, p. 890, 2017.

24. F. Kovacs, M. Torok, and I. Habermajer, “A rule-based phonocardiographic method for long-term fetal heart rate monitoring,” IEEE Trans. Biomed. Eng., vol. 47, no. 1, pp. 124-130, Jan. 2000.

25. D. G. Talbert, W. L. Davies, F. Johnson, N. Abraham, N. Colley, and D. P. Southall, “Wide bandwidlt fetal phonography using a sensor matched to the compliance of the mother's abdominal wall,” IEEE Trans. Biomed. Eng., vol. BME-33, no. 2, pp. 175-181, Feb. 1986.

26. V. Padmanabhan, J. L. Semmlow, and W. Welkowitz, “Accelerometer type cardiac transducer for detection of low-level heart sounds,” IEEE Trans. Biomed. Eng., vol. 40, no. 1, pp. 21-28, Jan. 1993.

27. K. Nishihara, S. Horiuchi, H. Eto, and M. Honda, “A long-term monitoring of fetal movement at home using a newly developed sensor: An introduction of maternal micro-arousals evoked by fetal movement during maternal sleep,” Early Hum. Develop., vol. 84, no. 9, pp. 595-603, 2008.

28. B. Boashash, M. S. Khlif, T. Ben-Jabeur, C. E. East, and P. B. Colditz, “Passive detection of accelerometer-recorded fetal movements using a time-frequency signal processing approach,” Digit. Signal Process., vol. 25, pp. 134-155, Feb. 2014.

29. M. Altini, “Detection of fetal kicks using body-worn accelerometers during pregnancy: Trade-offs between sensors number and positioning,” in Proc. 38th Annu. Int. Conf. IEEE Eng. Med. Biol. Soc. (EMBC), Aug. 2016, pp. 5319-5322.

30. M. Mesbah, M. S. Khlif, C. East, J. Smeathers, P. Colditz, and B. Boashash, “Accelerometer-based fetal movement detection,” in Proc. Annu. Int. Conf. IEEE Eng. Med. Biol. Soc., Aug./Sep. 2011, pp. 7877-7880.

31. M. Etemadi and O. T. Inan, “Wearable ballistocardiogram and seismocardiogram systems for health and performance,” J. Appl. Physiol., vol. 124, no. 2, pp. 452-461, 2018.

32. C. Yang and N. Tavassolian, “Combined seismo- and gyro-cardiography: A more comprehensive evaluation of heart-induced chest vibrations,” IEEE J. Biomed. Health Inform., vol. 22, no. 5, pp. 1466-1475, Sep. 2018.

33. M. J. Tadi et al., “Gyrocardiography: A new non-invasive monitoring method for the assessment of cardiac mechanics and the estimation of hemodynamic variables,” Sci. Rep., vol. 7, Jul. 2017, Art. no. 6823.

34. (2019). Shimmer Sensing. Accessed: Jul. 30, 2019. [Online]. Available: https://www.shimmersensing.com

35. F. Bousefsaf, C. Maaoui, and A. Pruski, “Continuous wavelet filtering on webcam photoplethysmographic signals to remotely assess the instantaneous heart rate,” Biomed. Signal Process. Control, vol. 8, no. 6, pp. 568-574, 2013.

36. Y. Zhang et al., “Motion artifact reduction for wrist-worn photoplethysmograph sensors based on different wavelengths,” Sensors, vol. 19, no. 3, p. 673, Feb. 2019.

37. A. Taebi and H. A. Mansy, “Time-frequency distribution of seismocardiographic signals: A comparative study,” Bioengineering, vol. 4, no. 2, p. 32, Jun. 2017.

38. C. Bruser, J. M. Kortelainen, S. Winter, M. Tenhunen, J. Parkka, and S. Leonhardt, “Improvement of force-sensor-based heart rate estimation using multichannel data fusion,” IEEE J. Biomed. Health Inform., vol. 19, no. 1, pp. 227-235, Jan. 2015.

39. T. Y. Euliano et al., “Monitoring fetal heart rate during labor: A comparison of three methods,” J. Pregnancy, vol. 2017, Mar. 2017, Art. no. 8529816.

40. W. R. Cohen et al., “Accuracy and reliability of fetal heart rate monitoring using maternal abdominal surface electrodes,” Acta Obstetricia Gynecologica Scandinavica, vol. 91, no. 11, pp. 1306-1313, 2016.

41. American Academy of Pediatrics, American College of Obstetricians and Gynecologists Fetal Heart Rate Monitoring, Guidelines for Perinatal Care, 7th ed. Washington, D.C., USA: American College of Obstetricians and Gynecologists, 2012.

What is claimed is:

1. A system for detecting fetal heart rate (FHR) of a subject, comprising:
a plurality of inertial measurement units (IMUs) configured to detect the FHR based on at least one of (i) a seismo-cardiogram (SCG) signal, or (ii) a gyro-cardiogram (GCG) signal received from at least one portion the subject.

2. The system of claim 1, further comprising:
a plurality of acoustic sensors configured to detect the FHR.

3. The system of claim 2, wherein the plurality of acoustic sensors are configured to forward beam-formed signals provided thereby.

4. The system of claim 1, further comprising:
at least one electrocardiogram (ECG) sensor is configured to detect the FHR.

5. The system of claim 4, wherein the at least one ECG sensor is a single-lead ECG sensor.

6. The system of claim 1, wherein each of the plurality of IMUs includes an accelerometer and a gyroscope.

7. The system of claim 1, wherein the plurality of IMUs are configured to detect a movement of the subject.

8. A system for detecting fetal heart rate (FHR) of at least one subject, comprising:
at least one sensor configured to detect the FHR, wherein the at least one sensor comprises at least one of:
a plurality of acoustic sensors which are configured to forward beam-formed signals provided thereby, or
at least one single-lead electrocardiogram (ECG) sensor.

9. The system of claim 8, further comprising:
a plurality of inertial measurement units (IMUS) which are configured to detect at least one of (i) the FHR or (ii) a fetal movement of the at least one subject.

10. The system of claim 8, further comprising:
at least one electrocardiogram (ECG) sensor configured to detect the FHR.

11. The system of claim 10, wherein the at least one ECG sensor is a single-lead ECG sensor.

12. The system of claim 8, wherein the at least one sensor comprises the plurality of acoustic sensors which are configured to forward the beam-formed signals provided thereby

13. A system for determining fetal heart rate (FHR) of at least one subject, comprising:
at least one electrocardiogram (ECG) sensor providing first signals or information regarding the at least one subject;
a plurality of inertial measurement units (IMUs) providing second signals or information regarding the at least one subject;
a plurality of acoustic sensors providing third signals or information regarding the at least one subject; and
at least one computer processor which is configured to determine data regarding the FHR based on the first, second and third signals or information.

14. The system of claim 13, wherein the at least one ECG sensor is a single-lead ECG sensor.

15. The system of claim 13, wherein the plurality of acoustic sensors are configured to provide beam-formed signals to the at least one subject.

16. The system of claim 13, wherein the IMUs which are configured to detect at least one of (i) the FHR or (ii) a fetal movement of the at least one subject.

17. A method for determining fetal heart rate (FHR) of at least one subject, comprising:
with at least one electrocardiogram (ECG) sensor, obtaining first signals or information regarding the at least one subject;
with a plurality of inertial measurement units (IMUs), obtaining second signals or information regarding the at least one subject;
with a plurality of acoustic sensors, obtaining third signals or information regarding the at least one subject; and
with a processor, determining data regarding the FHR based on the first, second and third signals or information.

18. The method of claim 17, wherein the at least one ECG sensor is a single-lead ECG sensor.

19. The method of claim 17, wherein the plurality of acoustic sensors are configured to provide beam-formed signals to the at least one subject.

20. The method of claim 17, wherein the IMUs which are configured to detect at least one of (i) the FHR or (ii) a fetal movement of the at least one subject.
